(19) United States
(12) Patent Application Publication
(10) Pub. No.: US 2026/0096936 A1
(43) Pub. Date: Apr. 9, 2026

(54) MULTI-LAYER URINE CAPTURE DEVICE AND RELATED METHODS

(71) Applicant: PureWick Corporation, Covington, GA (US)

(72) Inventors: Samuel Edmund Whittome, Cambridge (GB); Emily Lucy Triggs, Cambridge (GB); Gary Stacey, Cambridge (GB); Alexander David Norman, Cambridge (GB); Leanne Yip Heung Win, Middlesex (GB)

(21) Appl. No.: 19/418,150

(22) Filed: Dec. 12, 2025

Related U.S. Application Data
(63) Continuation of application No. 17/907,125, now Pat. No. 12,521,288.
(60) Provisional application No. 62/994,912, filed on Mar. 26, 2020.

(51) Int. Cl. A61F 13/537; A61F 13/15; A61F 13/53; A61M 1/00

(52) U.S. Cl. CPC A61F 13/5376; A61F 13/53704; A61F 13/53708; A61M 1/60; A61F 2013/15552; A61F 2013/530802

(57) ABSTRACT

Embodiments are directed to a multi-layered urine capture device suitable for collecting and transporting urine away from the body. The urine capture device can be positioned adjacent a urethra of a female user. The urine capture device can include a plurality of layers, for example, one or more fluid permeable layers, one or more absorbent layers, one or more hydrophobic layers, one or more wicking layers, one or more fluid impermeable layers, and other suitable layers.

### CROSS-REFERENCE TO RELATED APPLICATIONS

This application is a continuation of U.S. Application No. Ser. No. 17/907,125 filed on Sep. 23, 2022, which is a U.S. Nationalization of PCT International Application No. PCT/US2021/024162 filed on Mar. 25, 2021, which claims priority to U.S. Provisional Ser. No. 62/994,912 filed on Mar. 26, 2020, the disclosures of which are incorporated herein, in its entirety, by this reference.

### BACKGROUND

In various circumstances, a person may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues.

Females who suffer the most severe consequences of urinary incontinence are typically elderly and often bedbound. They also require continuous assistance to maintain hygiene. An insufficient or flawed device for reliably relocating urine away from the anatomy of the patient leads to rashes, sores, unhygienic conditions and other discomforts. For example, some devices leak or otherwise fail to retain captured urine in a manner which enables a dry hygienic interface between the anatomy of the user and the device. A multi-layered urine capture device should be designed with reference to the deficiencies of other devices.

### SUMMARY

Embodiments are directed to multi-layered urine capture devices suitable for collecting and transporting urine away from the body of a person. In an embodiment of the present disclosure, a multi-layered urine capture device to be positioned adjacent a urethra of a female user is disclosed. The multi-layered urine capture device includes a reservoir layer and a fluid impermeable layer. The reservoir layer can define a volume for receiving and storing urine discharged from a user. The fluid impermeable layer can define a bottom surface of the multi-layered urine capture device.

In another aspect of the present disclosure, a method for collecting discharged urine from a multi-layered urine capture device is disclosed. The method includes positioning the multi-layered urine capture device at least proximate to a urethra of a female user. The method also includes receiving discharged urine within a reservoir layer of the multi-layered urine capture device. The reservoir layer can be disposed between the urethra and a fluid impermeable layer of the multi-layered urine capture device. The reservoir layer defines a volume within the multi-layered urine capture device. The method also includes applying a suction force at a fluid conduit of the multi-layered urine capture device to draw urine out of the volume. The fluid conduit provides fluid communication between the multi-layered urine capture device and a suction source.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is an isometric view of a multi-layered urine capture device, according to an embodiment.

FIG. 2A is an exploded isometric view of the multi-layered urine capture device depicted in FIG. 1.

FIG. 2B is a cross-sectional view of the multi-layered urine capture device depicted in FIG. 1.

FIG. 3A is an exploded isometric view of a multi-layered urine capture device, according to another embodiment.

FIG. 3B is a cross-sectional view of the multi-layered urine capture device depicted in FIG. 3A.

FIG. 4A is an exploded isometric view of a multi-layered urine capture device, according to another embodiment.

FIG. 4B is a cross-sectional view of the multi-layered urine capture device depicted in FIG. 4A.

FIG. 5A is an exploded isometric view of a multi-layered urine capture device, according to another embodiment.

FIG. 5B is a cross-sectional view of the multi-layered urine capture device depicted in FIG. 5A.

FIG. 6A is an exploded isometric view of a multi-layered urine capture device, according to another embodiment.

FIG. 6B is a cross-sectional view of the multi-layered urine capture device depicted in FIG. 6A.

FIG. 7A is an exploded isometric view of a multi-layered urine capture device, according to another embodiment.

FIG. 7B is a cross-sectional view of the multi-layered urine capture device depicted in FIG. 7A.

FIG. 8 is an example flow diagram of a method for collecting discharged urine from a multi-layered urine capture device.

### DETAILED DESCRIPTION

Embodiments are directed to multi-layered urine capture devices suitable for collecting and transporting urine away from the body of a person. The multi-layered urine capture device can be positioned adjacent a urethra of a female user. The multi-layered urine capture device can include a plurality of layers, such as one or more fluid permeable layers, one or more absorbent layers, one or more hydrophobic layers, one or more wicking layers, one or more fluid impermeable layers, and so on. In some embodiments, the multi-layered urine capture device includes a reservoir layer and a fluid impermeable layer. The reservoir layer can define a volume for receiving and storing a liquid (e.g., urine). The fluid impermeable layer defines a volume within the device and a bottom surface of the multi-layered urine capture device. In some embodiments, the multi-layered urine capture device can also include a permeable layer. The permeable layer can be configured to direct urine away from a portion of skin surrounding a female anatomy of the user to provide a dry contact surface. The reservoir layer can be positioned between the permeable layer and the fluid impermeable layer. The permeable layer can bias the liquid to remain within the reservoir layer. In some embodiments, the multi-layered urine capture device can include one or more fluid conduits positioned adjacent the reservoir layer. The one or more fluid conduits can be configured to extract the liquid from the multi-layered urine capture device. The fluid conduit can be operably coupled to a vacuum source which extracts urine from the multi-layered urine capture device. One suitable non-limiting example of a vacuum source that can be used is the DryDoc Vacuum Station, available from PureWick, Inc.

The plurality of layers can be situated in various combinations which one or more of: prevent or reduce leakage, promote an adjustable fit (i.e., variable contour to fit a patient's of varying dimensions), or reduce or eliminate odor. The varying layers described herein can be adhered, stitched, welded, fastened (e.g., hook and loop fastener), or otherwise coupled to one another. The layers can also be stacked or ordered in various combinations so long as the functionality of the layer is maintained.

In some embodiments, the multi-layered urine capture device can be positioned between a user's (e.g., a patient) thighs to retain the device in a position abutting one or more regions about the anatomy of the user. For example, the multi-layered urine capture device can be positioned between the thighs of the user and proximal to the perineum of the user. In some embodiments, the width of the multi-layered urine capture device can vary from a proximal end to a distal end of the device. For example, a width of the multi-layered urine capture device positioned between the user's thighs can be narrower than a width of the device positioned proximal to the user's buttocks. In other embodiments, the width of the multi-layered urine capture device can remain constant or substantially constant from the proximal end to the distal end.

In some embodiments, the multi-layered urine capture device can be used in conjunction with a pump or vacuum system which applies a negative pressure within the device to extract urine from the device (e.g., the reservoir layer, the volume defined by the fluid impermeable layer, etc.). In some embodiments, the device can also be used in conjunction with a plurality of sensors disposed within the device, such as on or in the fluid impermeable layer. The sensors can be configured to take measurements relative to the presence of urine. For example, sensors can measure pH values, protein concentrations, moisture levels, and so on. The sensors can be coupled (wirelessly or wired) to a computing device or other ancillary system configured to receive the measured information. For example, a detected or measured moisture level within the device that exceeds a predetermined threshold can be received by a vacuum system and trigger the system to initiate and extract urine from the device.

In some embodiments, the multi-layered urine capture device includes a fluid conduit, such as a tube disposed adjacent the reservoir layer. In other embodiments, the device may not include a tube but is configured to receive a tube for extracting urine from the device. The fluid impermeable layer can include electrical traces and house one or more sensors configured to take various measurements (e.g., moisture sensors, protein sensors, pH sensors, etc.). The one or more sensors can cause an ancillary device to operate, for example, moisture detected by a moisture sensor can cause a vacuum system to operate.

Any of layers described herein, including the adjustment mechanism, can be used in any combination to comprise the multi-layered urine capture device. In other words any single layer described herein can be used in combination with one or more of any other layer(s) described herein. Furthermore, the position of any one layer relative to any other layer described herein is variable. In other words, any single layer described herein can be positioned between, above, or below any other layer described herein. Accordingly, the specific embodiments described herein and shown in the figures should not be construed as limiting the totality of possible combinations of layers. Other layers and materials not specifically mentioned in this disclosure but known to persons having skill in the art, such as airlaid paper, Teflon, adhesives, and so on, are contemplated by this disclosure.

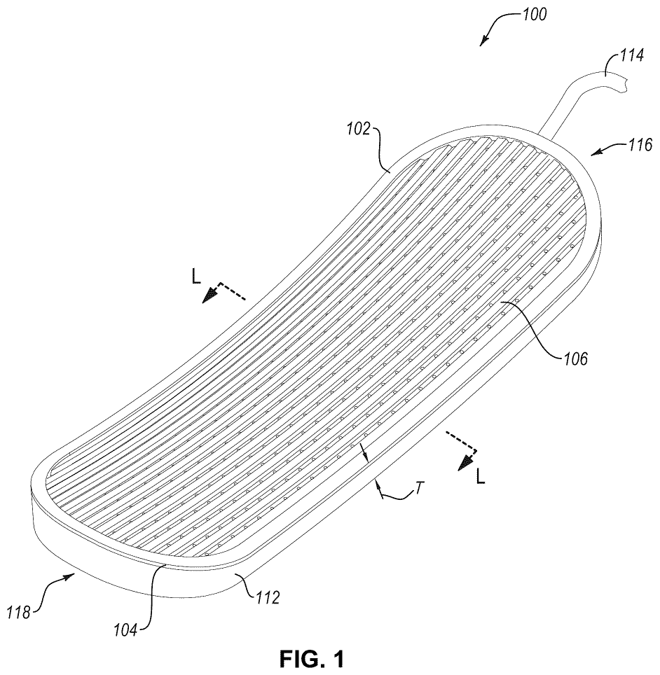

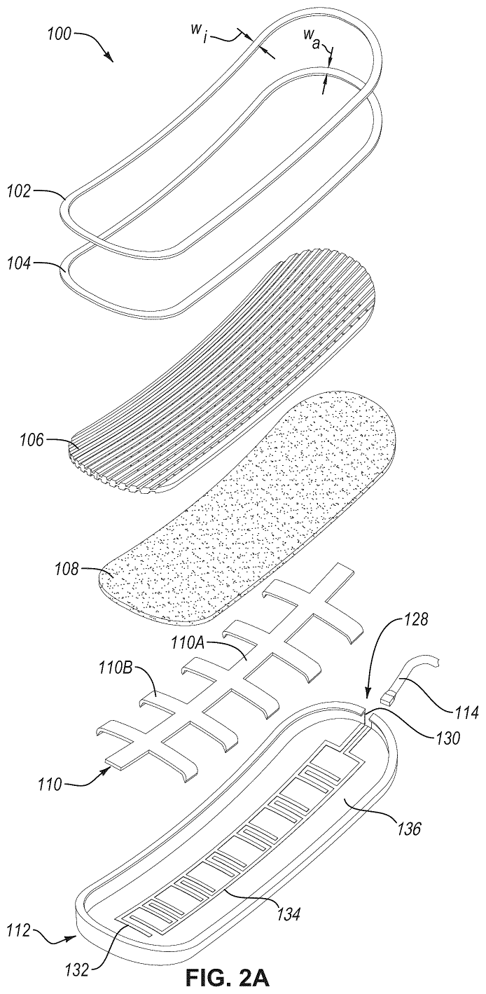

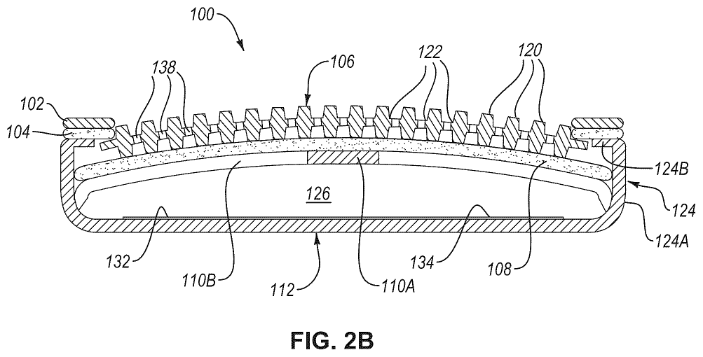

FIG. 1 is an isometric view of a multi-layered urine capture device 100, according to an embodiment. The multi-layered urine capture device 100 includes an interface layer 102, an air inlet layer 104, a permeable layer 106, a reservoir layer 108 (not shown, see FIGS. 2A and 2B), an adjustment mechanism 110 (not shown, see FIGS. 2A and 2B), a fluid impermeable layer 112, and a fluid conduit 114. The device 100 has a proximal end 116 and a distal end 118. In examples, the fluid conduit 114 can be positioned at the proximal end 118 and adjacent to the reservoir layer 108 (not shown, see FIGS. 2A and 2B). A thickness T of the device 100 can vary along a length of the device 100. The thickness T can also vary relative to the number of layers that make up the device 100. Moreover, the thickness of each individual layer can vary from one embodiment to another. For example, the reservoir layer 108 can be thicker in a first embodiment and thinner in a second embodiment. In embodiments, each respective layer of the multi-layered urine capture device 100 can have a thickness of at least 0.1 millimeters, about 0.1 millimeters to about 1 millimeter, about 0.1 millimeters to about 2 millimeters, about 0.1 millimeters to about 5 millimeters, about 0.5 millimeters to about 1.5 millimeters, or less than 5 millimeters. As such, the thickness T of the device 100 can be at least .3 millimeters, about .5 millimeters to about 1 centimeter, about 1 millimeter to about 2 centimeters, or greater than 2 centimeters.

FIG. 2A is an exploded isometric view of the multi-layered urine capture device 100 depicted in FIG. 1. The multi-layered urine capture device 100 includes the interface layer 102, the air inlet layer 104, the permeable layer 106, the reservoir layer 108, the adjustment mechanism 110, the fluid impermeable layer 112, and the fluid conduit 114.

In some embodiments, the interface layer 102 is the uppermost layer of the multi-layered urine capture device 100. The interface layer 102 can maintain a sealed or leak-resistant barrier between the patient's body and the multi-layered urine capture device 100. The interface layer 102 can expand or contract to mold against the skin of the user. For example, in some embodiments, the interface layer 102 can be a hydrocolloid strip which forms a gel barrier when exposed to liquid (e.g., urine). Although adhesives may be utilized as an interface layer, a gel barrier is less invasive than the use of adhesives which can irritate the skin of the user.

In some embodiments, the interface layer 102 can define an uppermost surface of the multi-layered urine capture device 100. As depicted in FIGS. 1, 2A, and 2B, the interface layer 102 can form a wall that extends around or substantially around the periphery of the multi-layered urine capture device 100. In some embodiments, the interface layer 102 can define a single continuous wall extending around the periphery of the device 100. In other embodiments, the interface layer 102 can include distinct discontinuous sections positioned around the periphery of the device 100.

The interface layer 102 can define a width Wᵢ. The width Wᵢ of the interface layer 102 can vary about the periphery of the device 100. For example, the interface layer 102 can be wider in sections exposed to more urine (e.g., sections of the interface layer 102 directly adjacent the user's urethra opening). Similarly, the thickness of the interface layer 102 can be constant about the periphery of the device 100, or vary about the periphery of the device 100 relative to urine exposure. For example, the interface layer 102 can be thicker in sections exposed to more urine (e.g., sections of the interface layer 102 directly adjacent the user's urethra opening).

In some embodiments, the interface layer 102 can be made of one or more non-absorbent material such as, gel, foam, natural and synthetic polymers, natural and synthetic fibers, and so on. For example, at least a portion of the interface layer 102 can be made of polytetrafluoroethylene (PTFE). As such, the interface layer 102 can be non-absorbent or substantially non-absorbent and thereby prevent urine from leaking or seeping out of the device 100 where the device 100 contacts the user's skin.

The air inlet layer 104 can be positioned between the interface layer 102 and another layer of the multi-layered urine capture device 100. For example, as shown in FIG. 2B, the air inlet layer 104 can be positioned between the interface layer 102 and the fluid impermeable layer 112 (e.g., the horizontal portion 124B of the wall 124 of the fluid impermeable layer 112). In some embodiments, the air inlet layer 104 allows air to travel into and out of the multi-layered urine capture device 100 and thereby circulate air relative to the device 100. Circulating air into and out of the device 100 will allow wet or damp portions of the user's skin to dry and thereby avoid rashes, sores, odors, hygienic issues, and other problems associated with prolonged exposure to urine. The air inlet layer 104 can be a foam or other venting material (e.g., Porex®, Gore-Tex®, etc.) that allows air to flow into and out of the device 100. In some embodiments, the air inlet layer 104 can be positioned under the interface layer 102 and extend around the periphery of the multi-layered urine capture device 100. The air inlet layer 104 can be positioned above the permeable and reservoir layers such that the permeable layer prevents or inhibits urine temporarily stored within the reservoir layer from leaking out of the air inlet layer 104.

In some embodiments, the air inlet layer 104 and the interface layer 102 can be a single layer. For example, the interface layer 102 can include at least one portion or section that is configured to allow air to travel into and out of the device 100 while also preventing liquid (e.g., urine) from leaking out of the device 100. A first and second material can be combined to produce a material which prevents liquid from leaking out of the device while also permitting air to vent relative to the device 100. Additionally or alternatively, a single material can be used which vents air while simultaneously preventing leaks (e.g., Gore-Tex®).

Similar to the interface layer 102, the air inlet layer 104 can define a width Wₐ. The width Wₐ of the air inlet layer 104 can vary about the periphery of the device 100. Moreover, the thickness of the air inlet layer 104 can be constant about the periphery of the device 100, or vary about the periphery of the device 100.

The air inlet layer 104 and/or the interface layer 102 can form the sides of a cavity or volume above the permeable layer 106. The air inlet layer 104 can be configured to permit air to flow into and out of the cavity or volume. The cavity or volume can space the permeable layer 106 apart from the anatomy of the user by a distance equal to the thicknesses of the air inlet layer 104 and/or interface layer 102. Such spacing can be beneficial in preventing or inhibiting unnecessary contact between the user's anatomy and the device 100 and thereby reduce potential rashes, sores, or discomfort caused by unnecessary contact.

Generally, the permeable layer 106 can be positioned between the air inlet layer 104 and the reservoir layer 108. As shown in FIG. 2B, the air inlet layer 104 can cover or overlap a portion of the permeable layer 106 in some embodiments. The permeable layer 106 can contact a portion of the user's anatomy while in use. As such, in some embodiments, the permeable layer 106 can be hydrophobic and therefore direct urine away from the user's body to provide a substantially moisture-free contact surface. The permeable layer 106 can bias the urine to remain within or below the reservoir layer 108. The permeable layer 106 can include liquid-directing features, such as, hydrophobic ridges 120 and channels 122. The liquid directing features direct urine through apertures 138 within the channels 122. The apertures 138 within the channels 122 direct urine to subsequent layers of the multi-layered urine capture device 100. Additionally or alternatively, the permeable layer 106 can receive one or more hydrophobic coatings, such as, a silica-based coating or oxide polystyrene composite coating.

The liquid directing features (e.g., hydrophobic ridges 120) can protrude from the permeable layer 106 into a volume or cavity within the multi-layered urine capture device 100 formed by the combined thickness of an interface layer 102 and an air inlet layer 104. Additionally or alternatively, the liquid directing features (e.g., hydrophobic ridges 120) can protrude from the permeable layer 106 toward an underlying layer, such as, the reservoir layer 108. The permeable layer 106 can be a singular molded or extruded layer. Alternatively, the permeable layer 106 can include multiple layers, such as, a base layer to which the liquid directing features are subsequently attached or adhered. For example, a plurality of hydrophobic ridges can be adhered to a non-absorbent base layer to generate the permeable layer 106.

The permeable layer 106 can cover or overlay one or more other layers of the device 100. The permeable layer 106 can be non-abrasive to mitigate or prevent irritation to any portion of the patient's skin which contacts the permeable layer 106. The permeable layer 106 can include multiple layers of material, such as, hydrophobic or wicking layers having varied permeable properties. In some embodiments, the permeable layer 106 can be formed from or include one or more porous non-absorbent materials, such as polytetrafluoroethylene (PTFE), nanocarbons, oleophobic materials, or oleophobic coatings.

In other embodiments, the permeable layer 106 can include any material that can wick fluid. For example, the permeable layer 106 can include fabric, such as a gauze (e.g., a silk, linen, polyester, or cotton gauze), another soft fabric (e.g., jersey knit fabric or the like), or another smooth fabric (e.g., rayon, satin, or the like). In some examples, the permeable layer 106 can include an open cell foam. Forming the permeable layer 106 from gauze, soft fabric, and/or smooth fabric can reduce chafing caused by contacting the user's skin. The permeable layer 106 can additionally or alternatively be formed using one or more layers of polytetrafluoroethylene (PTFE) and/or spun plastic fibers (e.g., spun nylon fibers).

As described herein, one or more of the layers, including the permeable layer 106, may include fluid permeable material designed to wick or pass fluid therethrough. The permeable properties referred to herein may be wicking, capillary action, diffusion, or other similar properties or processes, and are referred to herein as “permeable” and/or “wicking. ” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt%, less than about 3 wt %, less than about 2 wt %, less than about 1 wt %, or less than about 0.5 wt % of the dry weight of the wicking material. Wicking material can include natural fibers. In such examples, the material may have a coating to prevent or limit absorption of fluid into the material, such as a water repellent coating.

In some embodiments, the permeable layer 106 can include portions which wick fluid while also including portions that are hydrophobic. For example, the permeable layer 106 can include a wicking material with a hydrophobic coating. The hydrophobic coating can be overlaid on an uppermost surface of the wicking material and thereby provide a moisture free contact surface for the wearer's anatomy. While the hydrophobic coating provides a moisture free contact surface, the wicking material can wick fluid away from the wearer's anatomy. The hydrophobic portion of the permeable layer 106 can be a coating over the wicking material in some embodiments. In other embodiments, the hydrophobic portion of the permeable layer 106 can include the hydrophobic ridges disclosed herein.

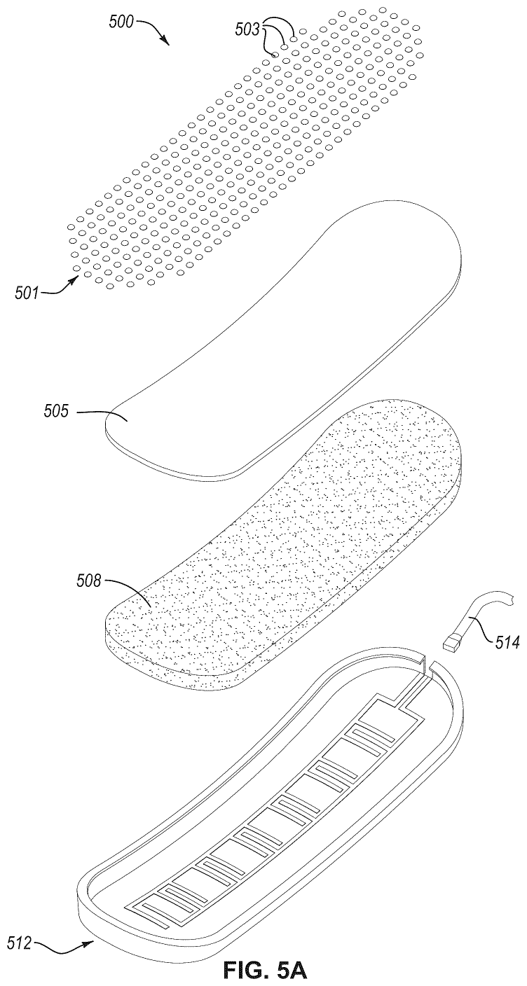

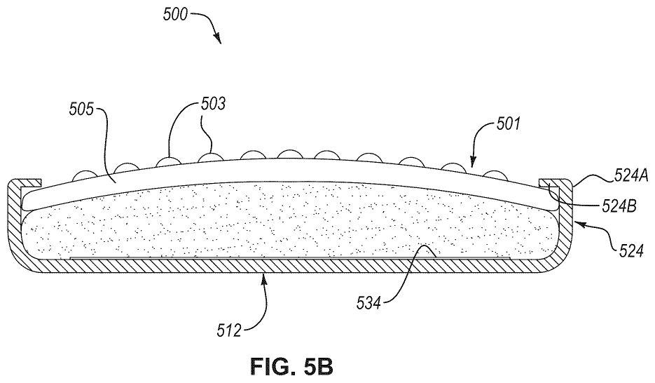

In some embodiments, the reservoir layer 108 can be positioned between the permeable layer 106 and the adjustment mechanism 110. In other embodiments such as those depicted in FIGS. 5A-7B, the reservoir layer can be positioned directly on the fluid impermeable layer 112. The reservoir layer 108 is a porous absorbent or semi-absorbent material that permits urine to pass therethrough. The reservoir layer 108 is also configured to form a volume within the device 100 in which urine can be temporarily stored (e.g., as the urine passes through the reservoir layer 108). The rate at which urine passes through the reservoir layer 108 can be controlled via the porosity, density or another characteristic of the layer. For example, the reservoir layer can be made up of a highly porous foam that permits urine to flow at a relatively high flow rate. In embodiments, a relatively higher flow rate can be at least 20 milliliters per second, about 20 milliliters per second to about 100 milliliters per second; about 30 milliliters per second to about 80 milliliters per second; about 40 milliliters per second to about 60 milliliters per second, or less than 100 milliliters per second. Alternatively, the reservoir layer 108 can be made up of a dense foam which permits urine to flow at a relatively low flow rate. In embodiments, a relatively low flow rate can be at least 1 milliliter per second, about 1 milliliter per second to about 40 milliliters per second; about 10 milliliters per second to about 20 milliliters per second; or less than 40 milliliters per second. The reservoir layer can be a foam, polyester (woven or nonwoven), or any other material which will create a volume within the device 100 to temporarily store the urine.

The adjustment mechanism 110 is configured to bend or flex to conform the multi-layered urine capture device 100 to users of varying sizes and dimensions. In some embodiments, the adjustment mechanism 110 includes a central member 110A and a plurality of lateral members 110B extending laterally (e.g., perpendicularly) from the central member 110A. The central member 110A can extend from the proximal end 116 to the distal end 118 of the device 100 and along a longitudinal axis of the device 100. The adjustment mechanism 110 can be a metallic frame capable of reconfigurably varying a contour of the multi-layered urine collection device 100. For example, the metallic frame can be formed from aluminum, an aluminum alloy, a shape memory material, plastic, wood, or other suitable material. In examples, the contour of the device 100 can substantially conform to a contour of the central member 110A of the adjustment mechanism 110.

The fluid impermeable layer 112 is configured to prevent urine from leaking from or propagating through the bottom of the multi-layered urine capture device 100. The fluid impermeable layer 112 can define the bottom or a bottom surface of the device 100. The bottom surface can have a contour which is reconfigurable by the adjustment mechanism 110. The fluid impermeable layer 112 can define at least a portion of the sides of the device 100. For example, the fluid impermeable layer 112 can form a wall 124 which defines a cavity 126 within the fluid impermeable layer 112. The fluid impermeable layer 112 can define a housing or enclosure wherein one or more layers of the device 100 are positioned within a volume 126 formed by the fluid impermeable layer 112.

The fluid impermeable layer 112 can be formed of any suitable fluid impermeable materials, such as a fluid impermeable polymer (e.g., silicone, polypropylene, polyethylene, polyethylene terephthalate, polyurethane, a polycarbonate, polyvinyl chloride, latex, silicone, etc.), a metal or alloy layer or film, another suitable material, or combinations thereof. In examples, the fluid impermeable layer 112 can be molded using an injection molding process. The fluid impermeable layer 112 can be relatively flexible or pliable such that reconfiguring the contour of the fluid impermeable layer 112 using the adjustment mechanism 110 does not break or damage the fluid impermeable layer 112. In some embodiments, the fluid impermeable layer 112 can be made of a material which has hydrophobic characteristics or is otherwise coated with a hydrophobic coating to reduce a quantity of residual urine within the device 100 after a vacuum source has extracted urine from the device 100.

The fluid impermeable layer 112 is the lowermost layer of the multi-layered urine capture device 100. The fluid impermeable layer 112 can include an outlet 128 configured to receive and retain the fluid conduit 114. The fluid conduit 114 can be a tube or fluid path fluidly coupled to a vacuum system for extracting the urine from the device 100. The outlet 128 can define a gap 130 or slot within the wall 124 of the fluid impermeable layer 112. The fluid conduit 114 can be affixed or mechanically retained within the gap 130 of the outlet 128. For example, the fluid conduit 114 can be adhered within the gap 130 of the outlet 128 such that the adhesive and fluid conduit 114 seal the gap 130 from leaking.

Alternatively or additionally, the outlet 128 can be configured to receive wiring operably coupled to one or more electrical traces 132, 134 within the fluid impermeable layer 112. The one or more electrical traces 132, 134 can be adhered, molded, or otherwise affixed to an inner surface 136 of the fluid impermeable layer 112. The one or more electrical traces 132, 134 can extend a length of the inner surface 136, for example, the electrical traces 132, 134 can extend a length of the inner surface 136 that spans substantially between the proximal end 116 and the distal end 118 of the device 100.

The electrical traces 132, 134 can communicatively couple one or more sensors (not shown) within the device 100 to the vacuum source or another instrument. For example, the sensors (not shown) within the device 100 may be configured to detect the presence of a liquid within the device 100 and cause the vacuum source to apply a negative pressure to the device 100 when liquid is detected by the sensors. Additionally or alternatively, the sensors can detect or measure pH values, protein concentrations, moisture levels, or other characteristics that may aid a physician in treating the user of the device 100.

FIG. 2B is a cross-sectional view of the multi-layered urine capture device 100 depicted in FIG. 1 taken through the section line L-L. The sectional view depicted in FIG. 2B illustrates the relative position of each layer with respect to the other layers of the device 100. For example, the interface layer 102 is the uppermost layer. The air inlet layer 104 is positioned directly below the interface layer 102. The permeable layer 106 is positioned below the air inlet layer 104. The reservoir layer 108 is positioned directly below the permeable layer 106. The adjustment mechanism 110 is positioned directly below the reservoir layer 108. The fluid impermeable layer 112 is the lowermost layer. While the permeable layer 106, the reservoir layer 108, and the adjustment mechanism 110 are illustrated in FIG. 2B as curved or arched, these layers can be flat or substantially planar in other embodiments.

The wall 124 of the fluid impermeable layer 112 can include a vertical portion 124A. The vertical portion 124A can define at least a portion of a side of the device 100. For example, as depicted in FIG. 2B, the vertical portion 124A encloses portions of the permeable layer 106, the reservoir layer 108, and the adjustment mechanism 110. In some embodiments, the wall 124 can also include a horizontal portion 124B. The horizontal portion 124B can partially extend between layers of the device 100. In the illustrated embodiment of the device 100 shown in FIG. 1, the horizontal portion 124B partially extends between the air inlet layer 104 and the permeable layer 108. In some embodiments, one or more layers of the device 100 can be position on or above the horizontal portion 124B and one or more layers of the device 100 can be positioned below or under the horizontal portion 124B. For example, the interface layer 102 and air inlet layer 104 can be positioned above the horizontal portion 124B while the permeable layer 106, reservoir layer 108, and the adjustment mechanism 110 are positioned below the horizontal portion 124B.

As described herein, the permeable layer 106 can include liquid directing features such as, hydrophobic ridges 120. The hydrophobic ridges 120 can define channels 122 within the permeable layer 106. The liquid directing features can be configured to direct the flow of urine into the reservoir layer 108. For example, the channels 122 can funnel urine to one or more apertures 138 or through-holes within the permeable layer 106. The liquid directing features can extend from a surface of the permeable layer 108 into a volume or cavity defined by the interface layer 102 and the air inlet layer 104. In examples, only the peak or crest of one or more of the hydrophobic ridges 120 contacts a portion of the user's anatomy such that a surface area of the permeable layer 106 that contacts the user's anatomy is reduced and thereby reduces potential discomfort caused by the contact. Alternatively or additionally, in some embodiments, at least a portion of the liquid directing features (e.g., one or more hydrophobic ridges 120) of the permeable layer 106 can extend toward the reservoir layer 108.

As illustrated in FIG. 2B, the fluid impermeable layer 112 can define a volume 126 or cavity. The adjustment mechanism 110 can define a top or upper surface of the volume 126. In some embodiments, each member of the plurality of lateral members 110B of the adjustment mechanism 110 can be bent or angled to define a portion of the volume 126 positioned below the adjustment mechanism 110. The volume 126 can store urine or other fluid discharged into the device 100. The reservoir layer 108 can also form a volume or semi-absorbent mass that permits urine to flow to the volume 126. The consistency, density, or other material property of the reservoir layer 108 can control a flow rate of urine through the reservoir layer 108. In embodiments including a reservoir layer 108 having a relatively low or slow flow rate, the reservoir layer 108 can act as a barrier preventing urine from exiting the volume through the reservoir layer 108 yet also permit urine to flow into the volume 126. In other words, gravitational forces and the flow rate of the reservoir layer 108 can work in conjunction to permit urine to pass through the reservoir layer 108 and into the volume 126 while simultaneously preventing urine from travelling back out of the volume 126 and through the reservoir layer 108.

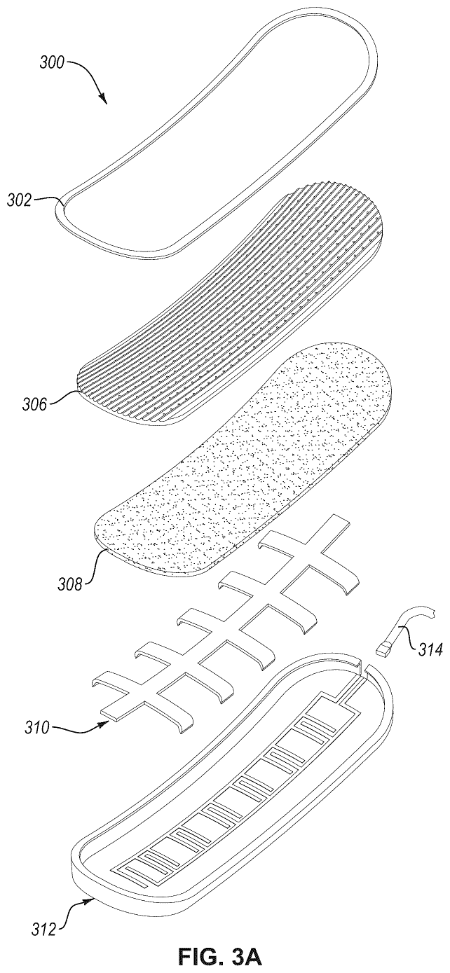

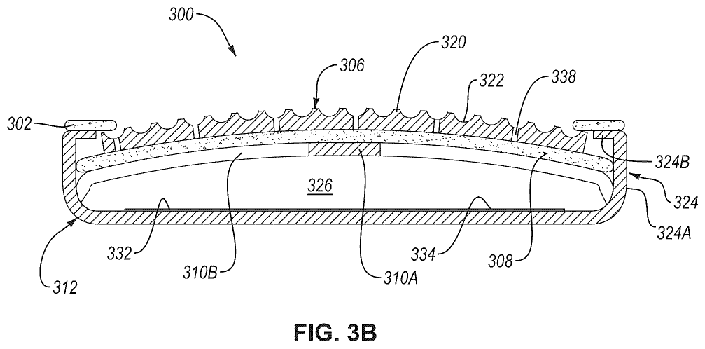

FIG. 3A is an exploded isometric view of a multi-layered urine capture device 300, according to another embodiment. The embodiment of the multi-layered urine capture device 300 includes an interface layer 302, a permeable layer 306, a reservoir layer 308, an adjustment mechanism 310, a fluid impermeable layer 312, and a fluid conduit 314. Each of the layers 302, 306, 308, 310, and 312 can include the characteristics and attributes of their respective layers described above with respect to FIGS. 1, 2A, and 2B. For example, the permeable layer 306 can be substantially the same or similar as the permeable layer 106 described above. Furthermore, the fluid conduit 314 can include the characteristics and attributes of the fluid conduit 114 described above with respect to FIGS. 1, 2A, and 2B.

The multi-layered urine capture device 300 is similar to the device 100 depicted in FIGS. 1-2B with the exception that the interface layer 102 and air inlet layer 104 of FIGS. 1-2B have been replaced by the singular interface layer 302. As such, the interface layer 302 can have the attributes of a combination of the interface layer 102 and air inlet layer 104 described herein. In other words, the interface layer 302 can act as a breathable barrier that is flexible and configured to conform to the skin of a user to generate a liquid barrier between the user's skin and the multi-layered urine capture device 300. Combining the interface layer 102 and the air inlet layer 104 into a single layer (i.e., interface layer 302) can minimize potential leaks within the device 300. For example, eliminating an interface between layers (i.e., the interface between the interface layer 102 and the air inlet layer 104) can reduce regions for potential leaks. Combining the layers can also lower production costs, manufacturing times, and reduce inventory.

FIG. 3B is a cross-sectional view of the multi-layered urine capture device 300. The section view in FIG. 3B is taken through a mid-portion of the multi-layered urine capture device 300 (similar to the location of the section line L-L shown in FIG. 1).

In this embodiment, the permeable layer 306 is different than the permeable layer 106 of the embodiment shown in FIGS. 1, 2A, and 2B. More specifically, the permeable layer 306 includes liquid directing features (e.g., hydrophobic ridges 320, channels 322, and apertures 338) formed on a surface facing the user's anatomy and does not include liquid directing features formed on a surface that interfaces with the reservoir layer 308. However, like the permeable layer 106 depicted in FIGS. 1-2B, the permeable layer 306 also includes hydrophobic ridges 320, channels 322, and apertures 338 that direct urine to reservoir layer 308 and therefore provides substantially similar functionality as the permeable layer 106 described herein.

Although the hydrophobic ridges 320 are illustrated as extending lengthwise (i.e., from the distal end to the proximal end of the device), the hydrophobic ridges 320 can additionally or alternatively extend between respective sides of the device. Furthermore, although the hydrophobic ridges 320 are illustrated as substantially linear or parallel, the ridges 320 can form a zig-zag pattern, square grid pattern, a triangular grid pattern, or the like.

Like the fluid impermeable layer 112, the fluid impermeable layer 312 can define the bottom or a bottom surface of the device 300. The bottom surface can have a contour which is reconfigurable by the adjustment mechanism 310. One or more traces 332, 334 can be positioned on a surface of the fluid impermeable layer 312 to utilize sensors within the device 300. The fluid impermeable layer 312 can define at least a portion of the sides of the device 300. For example, the fluid impermeable layer 312 can form a wall 324 which defines a cavity or volume 326. The fluid impermeable layer 112 can define a housing or enclosure wherein one or more layers of the device 300 are positioned within the cavity or volume 326. The wall 324 of the fluid impermeable layer 312 can include a vertical portion 324A. Optionally, the wall 324 can also include a horizontal portion 324B. The horizontal portion 324B can partially extend between layers of the device 300. In the embodiment of the device 300 shown in FIGS. 3A and 3B, the horizontal portion 324B partially extends between the interface layer 302 and the permeable layer 306.

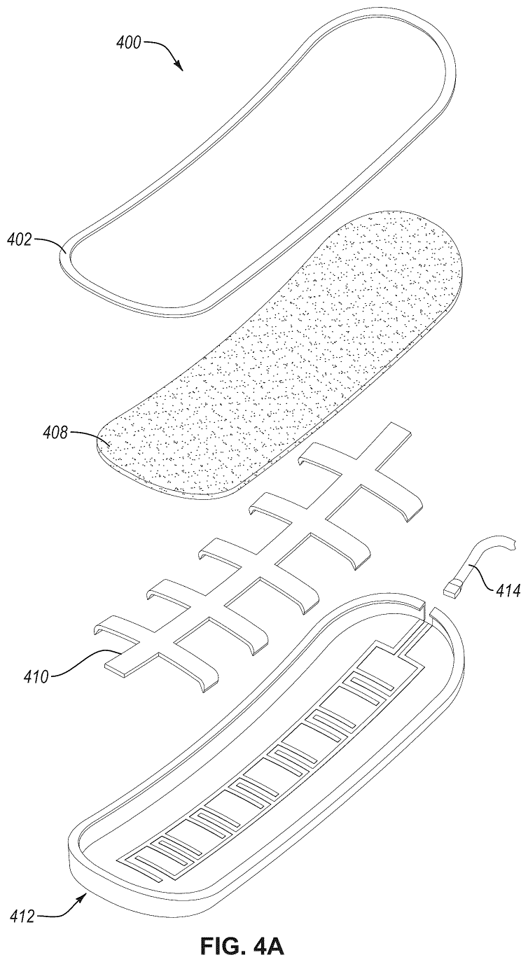

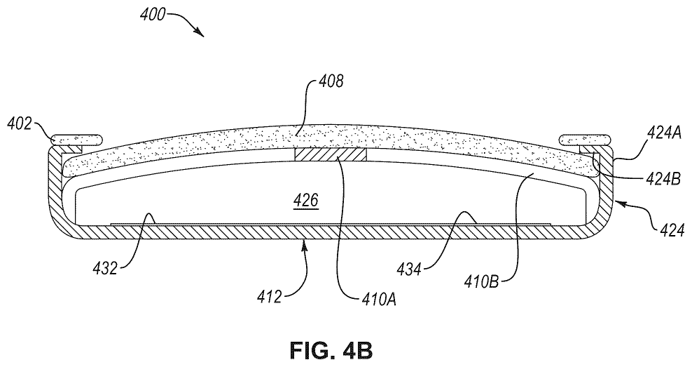

FIG. 4A is an exploded isometric view of a multi-layered urine capture device 400, according to another embodiment. The embodiment of the multi-layered urine capture device 400 includes an interface layer 402, a reservoir layer 408, an adjustment mechanism 410, a fluid impermeable layer 412, and a fluid conduit 414. The embodiment of the device 400 illustrated in FIGS. 4A and 4B differ from the previous embodiments depicted in FIGS. 1-3B. For example, FIGS. 4A and 4B do not include a permeable layer positioned between the interface layer 402 and the reservoir layer 408. Each of the layers 402, 408, 410, and 412 can include the characteristics and attributes of their respective layers described above with respect to FIGS. 1-3B. For example, the reservoir layer 408 can be substantially similar to or the same as the reservoir layer 108 described above. Furthermore, the fluid conduit 414 can include the characteristics and attributes of the fluid conduit described above with respect to FIGS. 1-3B.

Like the interface layer 102, the interface layer 402 can maintain a sealed or leak-resistant barrier between the patient's body and the multi-layered urine capture device 400. Alternatively or additionally, like the interface layer 302, the interface layer 402 can also allow air to travel into and out of the multi-layered urine capture device 400 and thereby circulate air relative to the device 400.

FIG. 4B is a cross-sectional view of the multi-layered urine capture device 400. The section view in FIG. 4B is taken through a mid-portion of the multi-layered urine capture device 400 (similar to the location of the section line L-L shown in FIG. 1).

In some embodiments, the reservoir layer 408 can interface or otherwise contact a portion of the user's anatomy while the device 400 is in use (along with the interface layer 402). In such embodiments, at least a portion of the reservoir layer 408 can be made of a non-absorbent wicking material to provide a moisture free contact surface. Additionally or alternatively, the reservoir layer 408 can receive one or more hydrophobic coatings, such as, a silica-based coating or oxide polystyrene composite coating. As described herein, the reservoir layer 408 can retain urine within the device 400 by regulating or otherwise dictating the flow rate at which urine can flow into and out of a volume 426 defined by the fluid impermeable layer 412. The reservoir layer 408 can be made using a dense semi-absorbent foam such that the reservoir layer 408 has a relatively low flow rate. A relatively low flow rate, along with gravitational forces, can inhibit urine from travelling back up through the reservoir layer 408 from the volume 426 thereby effectively preventing urine from leaking through an upper surface of the device 400.

Like the fluid impermeable layer 112, the fluid impermeable layer 412 can define the bottom or a bottom surface of the device 400. The bottom surface can have a contour which is reconfigurable by the adjustment mechanism 410. One or more traces 432, 434 can be positioned on a surface of the fluid impermeable layer 412 to accommodate the use of one or more sensors within the device 400. The fluid impermeable layer 412 can define at least a portion of the sides of the device 400. For example, the fluid impermeable layer 412 can form a wall 424 which defines the cavity or volume 426. The fluid impermeable layer 412 can define a housing or enclosure wherein one or more layers of the device 400 are positioned within the cavity or volume 426. The wall 424 of the fluid impermeable layer 412 can include a vertical portion 424A. Optionally, the wall 424 can also include a horizontal portion 424B. The horizontal portion 424B can partially extend between layers of the device 400. In the embodiment of the device 400 shown in FIGS. 4A and 4B, the horizontal portion 424B partially extends between the interface layer 402 and the reservoir layer 408.

FIG. 5A is an exploded isometric view of a multi-layered urine capture device 500, according to another embodiment. The embodiment of the multi-layered urine capture device 500 includes a dimple layer 501, a one-way fluid permeable layer 505, a reservoir layer 508, a fluid impermeable layer 512, and a fluid conduit 514. The embodiment of the device 500 illustrated in FIGS. 5A and 5B differ from the previous embodiments depicted in FIGS. 1-4B. For example, FIGS. 5A and 5B include the dimple layer 501 and the one-way fluid permeable layer 505 instead of the permeable layer of FIGS. 1-3B. Moreover, the embodiment of FIGS. 5A and 5B do not include the interface layer, the air inlet layer, or the adjustment mechanism. Each of the layers 502, 508 and 512 can include the characteristics and attributes of their respective layers described above with respect to FIGS. 1-4B. For example, the reservoir layer 508 can be substantially similar to or the same as the reservoir layer 108 described above. Furthermore, the fluid conduit 514 can include the characteristics and attributes of the fluid conduit 114 described above with respect to FIGS. 1-4B.

In embodiments, the multi-layered urine capture device 500 can include a dimple layer 501. The dimple layer 501 is positioned as the uppermost layer on the device 500. The dimple layer 501 pulls or wicks urine away from the user's body. The dimple layer 501 includes liquid directing features, such as dimples 503 that direct urine to subsequent layers of the multi-layered urine capture device 500 and provide a moisture-free barrier between the user's anatomy and the underlying layers (e.g., one-way fluid permeable layer 505). Each dimple 503 of the dimple layer 501 can be a porous non-absorbent material such as polytetrafluoroethylene (PTFE). The dimples 503 can protrude from another layer of the device 500. For example, the dimples 503 can protrude from the one-way fluid permeable layer 505. The dimples 503 can be adhered, welded, or otherwise coupled to another layer of the device 500 (e.g., the one-way fluid permeable layer 505). The dimple layer 501 can comprise liquid directing features (e.g., dimples 503, ribs, indentations, pits, protrusions, and so on). The liquid directing features can be displaced on the one-way fluid permeable layer 505 in a number of configurations. For example, the liquid directing features can be positioned on the upper surface of the one-way fluid permeable layer 505 in a dot-matrix wherein the spacing between each respective feature is consistent (as depicted in FIGS. 5A-B). In other embodiments, a higher density of features can be positioned near the centerline of the one-way fluid permeable layer 505 or near a respective end of the one-way fluid permeable layer 505.

In some embodiments, the multi-layered urine capture device 500 can include a one-way fluid permeable layer 505. The one-way fluid permeable layer 505 is positioned between the dimple layer 501 and the reservoir layer 508. The one-way fluid permeable layer 505 allows urine to flow through the one-way fluid permeable layer 505 to the reservoir layer 508 but prevents fluid from flowing back out of the reservoir layer 508. In other words, the one-way fluid permeable layer 505 can be made from a material that only permits unidirectional fluid flow (e.g., Nanotex). The one-way fluid permeable layer 505 can also be flexible and soft to provide a less invasive and more comfortable user contact surface.

FIG. 5B is a cross-sectional view of the multi-layered urine capture device 500. The section view in FIG. 5B is taken through a mid-portion of the multi-layered urine capture device 500 (similar to the location of the section line L-L shown in FIG. 1).

Like the fluid impermeable layer 112, the fluid impermeable layer 512 can define the bottom or a bottom surface of the device 500. One or more traces 532, 534 can be positioned on a surface of the fluid impermeable layer 512 to accommodate the use of one or more sensors within the device 500. The fluid impermeable layer 512 can define at least a portion of the sides of the device 500. For example, the fluid impermeable layer 512 can form a wall 524 which defines a cavity or volume within the fluid impermeable layer 512. In the embodiment shown in FIG. 5B, the reservoir layer 508 can occupy a substantial portion of the cavity or volume defined by the fluid impermeable layer 512. The wall 524 of the fluid impermeable layer 512 can include a vertical portion 524A. Optionally, the wall 524 can also include a horizontal portion 524B. The horizontal portion 524B can partially extend over layers of the device 500. In the embodiment of the device 500 shown in FIGS. 5A and 5B, the horizontal portion 524B partially extends over the one-way fluid permeable layer 505.

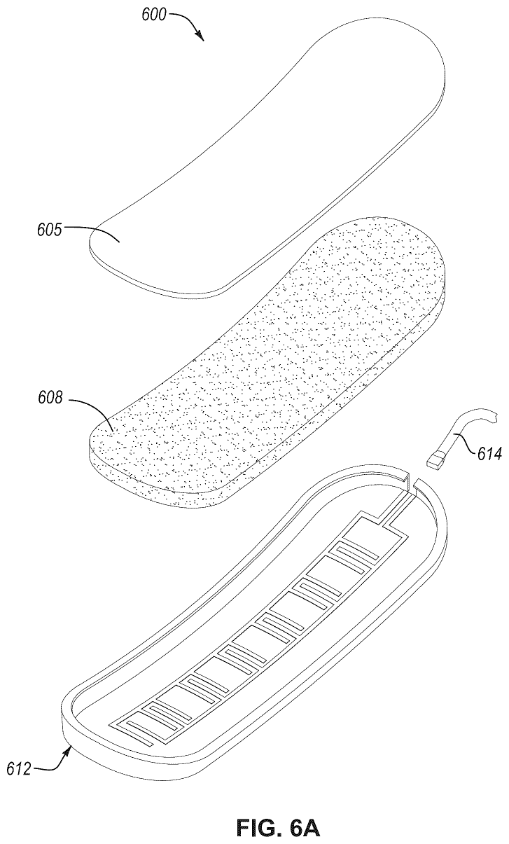

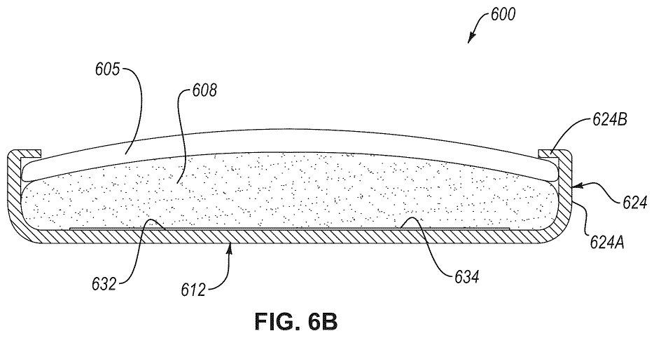

FIG. 6A is an exploded isometric view of a multi-layered urine capture device 600, according to another embodiment. The embodiment of the multi-layered urine capture device 600 includes a one-way fluid permeable layer 605, a reservoir layer 608, a fluid impermeable layer 612, and a fluid conduit 614. The embodiment of the device 600 illustrated in FIGS. 6A and 6B differ from the previous embodiments depicted in FIGS. 1-5B. For example, FIGS. 6A and 6B do not include the interface layer, the air inlet layer, the permeable layer, or the adjustment mechanism. Each of the layers 605, 608, and 612 can include the characteristics and attributes of their respective layers described above with respect to FIGS. 1, 2A, 2B, 5A, and 5B. For example, the one-way fluid permeable layer 605 can be substantially the same as the one-way fluid permeable layer 505 described above. Furthermore, the fluid conduit 614 can include the characteristics and attributes of the fluid conduit 114 described above with respect to FIGS. 1-5B.

In some embodiments, the one-way fluid permeable layer 605 is positioned as the uppermost layer of the device 600 and above the reservoir layer 608. Like the one-way fluid permeable layer 505, the one-way fluid permeable layer 605 allows urine to flow through the one-way fluid permeable layer 605 to the reservoir layer 608 but prevents fluid from flowing back out of the reservoir layer 608. In other words, the one-way fluid permeable layer 505 can be made from a material that only permits unidirectional fluid flow (e.g., Nanotex). As the one-way fluid permeable layer 605 can contact the user's anatomy in this embodiment, the one-way fluid permeable layer 605 can also be flexible and soft to provide a less invasive and more comfortable user contact surface.

FIG. 6B is a cross-sectional view of the multi-layered urine capture device 600. The section view in FIG. 6B is taken through a mid-portion of the multi-layered urine capture device 600 (similar to the location of the section line L-L shown in FIG. 1).

Like the fluid impermeable layer 112, the fluid impermeable layer 612 can define the bottom or a bottom surface of the device 600. One or more traces 632, 634 can be positioned on a surface of the fluid impermeable layer 612 to accommodate one or more sensors within the device 600. The fluid impermeable layer 612 can define at least a portion of the sides of the device 600. For example, the fluid impermeable layer 612 can form a wall 624 which defines a cavity or volume. In the embodiment shown in FIG. 6B, the reservoir layer 608 can occupy a substantial portion of the cavity or volume defined by the fluid impermeable layer 612. The wall 624 of the fluid impermeable layer 612 can include a vertical portion 624A. Optionally, the wall 624 can also include a horizontal portion 624B. The horizontal portion 624B can partially extend over layers of the device 600. In the embodiment of the device 600 shown in FIGS. 6A and 6B, the horizontal portion 624B partially extends over the one-way fluid permeable layer 605.

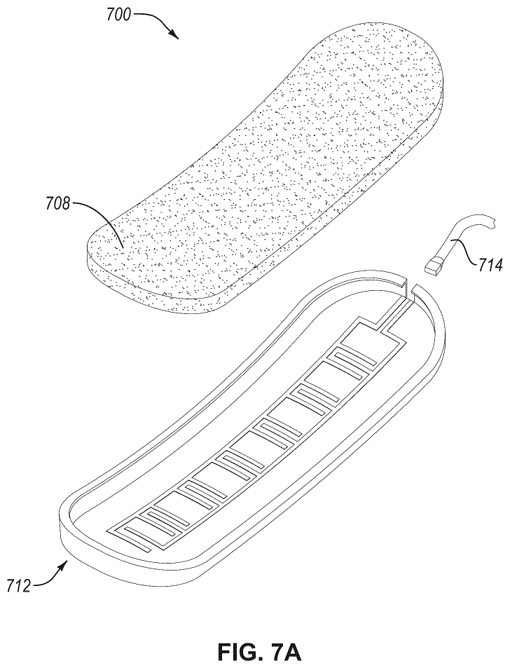

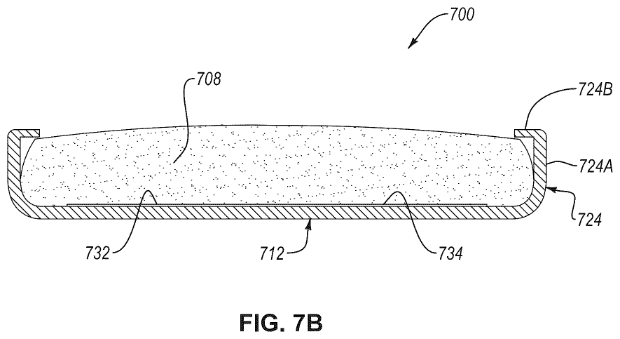

FIG. 7A is an exploded isometric view of a multi-layered urine capture device 700, according to another embodiment. The embodiment of the multi-layered urine capture device 700 includes a reservoir layer 708, a fluid impermeable layer 712, and a fluid conduit 714. The embodiment of the device 700 illustrated in FIGS. 7A and 7B differ from the previous embodiments depicted in FIGS. 1-6B. For example, FIGS. 7A and 7B illustrate a simplified embodiment that does not include many of the layer previously described herein. Instead, the embodiment illustrated in FIGS. 7A and 7B only includes the reservoir layer 708, the fluid impermeable layer 712, and the fluid conduit 714. Each of the layers 708 and 712 can include the characteristics and attributes of their respective layers described above with respect to FIGS. 1-6B. For example, the reservoir layer 708 can be substantially similar to or the same as the reservoir layer 108 described above. Furthermore, the fluid conduit 714 can include the characteristics and attributes of the fluid conduit 114 described above with respect to FIGS. 1, 2A, and 2B.

In this embodiment, the reservoir layer 708 may interface or otherwise contacts the user's anatomy while the device 700 is in use. As such, at least a portion of the reservoir layer 708 can be made of a non-absorbent wicking material to provide a moisture free contact surface. Additionally or alternatively, the reservoir layer 708 can receive one or more hydrophobic coatings, such as, a silica-based coating or oxide polystyrene composite coating. As described herein, the reservoir layer 708 can retain urine within the device 700 by regulating or otherwise dictating the flow rate at which urine can flow into and out of a volume defined by the fluid impermeable layer 712. The reservoir layer 708 can be made using a dense non-absorbent or semi-absorbent foam such that the reservoir layer 708 has a relatively low flow rate. A relatively low flow rate, along with gravitational forces, can inhibit urine from travelling back up through the reservoir layer 708 and thereby effectively prevent urine from leaking through an upper surface of the device 700.

FIG. 7B is a cross-sectional view of the multi-layered urine capture device 700. The section view in FIG. 7B is taken through a mid-portion of the multi-layered urine capture device 700 (similar to the location of the section line L-L shown in FIG. 1).

Like the fluid impermeable layer 112, the fluid impermeable layer 712 can define the bottom or a bottom surface of the device 700. One or more traces 732, 734 can be positioned on a surface of the fluid impermeable layer 712 to accommodate the use of one or more sensors within the device 700. The fluid impermeable layer 712 can define at least a portion of the sides of the device 700. For example, the fluid impermeable layer 712 can form a wall 724 which defines a cavity or volume. In the embodiment shown in FIG. 7B, the reservoir layer 708 can occupy a substantial portion of the cavity or volume defined by the fluid impermeable layer 712. The wall 724 of the fluid impermeable layer 712 can include a vertical portion 724A. Optionally, the wall 724 can also include a horizontal portion 724B. The horizontal portion 724B can partially extend over layers of the device 700. In the embodiment of the device 700 shown in FIGS. 7A and 7B, the horizontal portion 724B partially extends over the reservoir layer 708.

One or more of the layers described herein can be non-abrasive to mitigate or prevent irritation to any portion of the patient's skin which contacts the respective layer. One or more of the layers disclosed herein can also function as a deformable cushion or pad to provide additional comfort to the user. For example, in some embodiments, the permeable layer can be a foam, cloth, or other material which is non-abrasive and deformable. One or more of the layers described herein can be removable from the multi-layered urine capture device. For example, the reservoir layer can be coupled to the inner surface of the fluid impermeable layer using hook and loop fasteners or other mechanisms configured to allow removal without damaging the device.

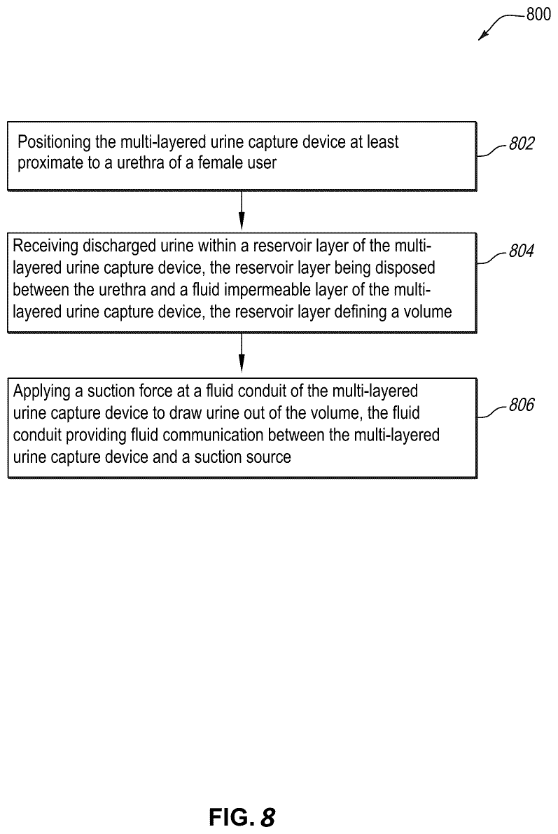

FIG. 8 illustrates an example method 800 for collecting discharged urine from a multi-layered urine capture device. The multi-layered urine capture device can be substantially similar to, and can include some or all of the features and/or components of the multi-layered urine capture devices described herein, such as multi-layered urine capture devices 100, 300, 400, 500, 600, 700. For example, the multi-layered urine capture device can include an interface layer, a permeable layer, a reservoir layer, an adjustment mechanism, a fluid impermeable layer, a fluid conduit, a dimple layer, and/or any other layer or component of the other multi-layered urine capture devices disclosed herein.

The method 800 includes the act 802 of positioning the multi-layered urine capture device at least proximate to a urethra of a female user. The method 800 includes the act 804 of receiving discharged urine within a volume defined by a reservoir layer of the multi-layered urine capture device. The method 800 includes the act 806 of applying a suction force at a fluid conduit of the multi-layered urine capture device to draw urine out of the volume. Accordingly, the method 800 can be utilized to collect urine discharged by a female user into the multi-layered urine capture device and extract the urine from the multi-layered urine capture device. The method 800 can include more acts than the acts 802-806. For example, once urine is expelled by the user, the method 800 can also include generating a liquid barrier between the periphery of the multi-layered urine capture device and the user's skin. The method 800 can also include directing expelled urine to channels or grooves within the permeable layer using the liquid directing features.

The method 800 includes the act 802 of positioning the multi-layered urine capture device at least proximate to a urethra of a female user. In other words, the multi-layered urine capture device can be disposed under and/or proximal to the genitals of a female user, such as adjacent the urethra opening or meatus of the female user. In some embodiments, the multi-layered urine capture device can include one or more layers that interface with the genitals of the female user, for example, an interface layer (e.g., interface layer 102, 302, 402).

The method 800 includes the act 804 of receiving discharged urine within a reservoir layer of the multi-layered urine capture device. The reservoir layer can be disposed between the urethra and a fluid impermeable layer of the multi-layered urine capture device. The reservoir layer can form or define a volume capable of storing a quantity of urine discharged into the multi-layered urine capture device. The reservoir layer can be substantially similar to or the same as the reservoir layers 108, 308, 408, 508, 608, 708 described above.

The method 800 includes the act 806 of applying a suction force at a fluid conduit of the multi-layered urine capture device to draw urine out of the volume. The fluid conduit can provide a pathway for fluid communication between the multi-layered urine capture device and a suction source. The suction source can be any vacuum or other device capable of applying a negative pressure to the multi-layered urine capture device to draw or extract urine through the fluid conduit.

While various embodiments of the multi-layered urine capture devices and methods have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular layers, features, and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

What is claimed is:

1. A urine capture device, comprising:
a fluid impermeable layer having a proximal end region, a distal end region, an inner surface at least partially defining a cavity, an outlet defined by the proximal end region, and an opening in fluid communication with the cavity; and
one or more electrical traces secured to the inner surface of the fluid impermeable layer, at least a portion of the one or more electrical traces being disposed at the distal end region of the fluid impermeable layer.

2. The urine capture device of claim 1, further comprising one or more sensors disposed within the urine capture device and coupled to the one or more traces, the one or more sensors being configured to detect urine, wherein at least a portion of the one or more electrical traces are disposed on the inner surface of the fluid impermeable layer generally opposite to the opening.

3. The urine capture device of claim 1, further comprising one or more fluid permeable members positioned at least partially within the cavity of the fluid impermeable layer, at least one of the one or more fluid permeable members extending at least partially across the opening.

4. The urine capture device of claim 3, wherein at least one of the one or more fluid permeable members is positioned on the inner surface of the fluid impermeable layer having at least a portion of the one or more electrical traces secured thereto.

5. The urine capture device of claim 3, wherein at least one of the one or more fluid permeable members includes a wicking material that is substantially non-absorbent.

6. The urine capture device of claim 1, wherein the fluid impermeable layer includes a rear region having the one or more electrical traces secured thereto, a front region positioned generally opposite to the rear region and at least partially defining the opening, and one or more side regions extending between the front region and the rear region.

7. The urine capture device of claim 6, further comprising one or more fluid permeable members positioned at least partially within the cavity of the fluid impermeable layer, at least a portion of the one or more fluid permeable layers being positioned between the front region and the rear region of the fluid impermeable layer, and at least one of the one or more fluid permeable members extending at least partially across the opening.

8. The urine capture device of claim 1, wherein the one or more electrical traces are adhered, molded, or affixed to the inner surface of the fluid impermeable layer, the one or more electrical traces being configured to connect the one or more sensors to an ancillary system configured to receive information from the one or more sensors.

9. The urine capture device of claim 1, further comprising a fluid conduit secured to the fluid impermeable layer and in fluid communication with the cavity through the outlet.

10. The urine capture device of claim 9, wherein the one or more electrical traces extend from the distal end region to the outlet, and wherein the outlet is configured receive wiring operably coupleable to the one or more electrical traces.

11. A urine capture device, comprising:
a fluid impermeable layer having a proximal end region defining, a distal end region, a rear region, a front region, an inner surface at least partially defining a cavity, an outlet defined by the proximal end region, and an opening defined by the front region and in fluid communication with the cavity; and
one or more electrical traces secured to the inner surface of the fluid impermeable layer, at least a portion of the one or more electrical traces being disposed on inner surface of the rear region of the fluid impermeable layer generally opposite to the opening.

12. The urine capture device of claim 11, further comprising one or more fluid permeable members positioned at least partially within the cavity of the fluid impermeable layer, at least a portion of the one or more fluid permeable layers being positioned between the front region and the rear region of the fluid impermeable layer, and at least one of the one or more fluid permeable members extending at least partially across the opening.

13. The urine capture device of claim 12, wherein at least one of the one or more fluid permeable members is positioned on the inner surface of the fluid impermeable layer having at least a portion of the one or more electrical traces secured thereto.

14. The urine capture device of claim 12, wherein at least one of the one or more fluid permeable members includes a wicking material that is substantially non-absorbent.

15. The urine capture device of claim 11, further comprising one or more sensors disposed within the urine capture device and coupled to the one or more traces, the one or more sensors being configured to detect urine, wherein the one or more electrical traces are adhered, molded, or affixed to the inner surface of the fluid impermeable layer, the one or more electrical traces being configured to connect the one or more sensors to an ancillary system configured to receive information from the one or more sensors.

16. The urine capture device of claim 11, further comprising a fluid conduit secured to the fluid impermeable layer and in fluid communication with the cavity through the outlet.

17. The urine capture device of claim 16, wherein the one or more electrical traces extend from the distal end region to the outlet, and wherein the outlet is configured receive wiring operably coupleable to the one or more electrical traces.

18. A method for collecting discharged urine with a capture device, the method comprising:
positioning a fluid permeable member of the urine capture device at least proximate to a urethra of a female user, the fluid permeable member extending at least partially across an opening of the urine collection device defined at least partially by a fluid impermeable layer of the urine capture device, wherein the fluid impermeable layer includes a proximal end region, a distal end region, an inner surface at least partially defining a cavity that is in fluid communication with the opening, and an outlet defined by the proximal end region, the fluid permeable member being positioned at least partially within the cavity;
receiving discharged urine within the cavity;
detecting the discharged urine in the cavity at with one or more sensors in the urine capture device, the one or more sensors being coupled to one or more electrical traces secured to the inner surface of the fluid impermeable layer, at least a portion of the one or more electrical traces being disposed at the distal end region of the fluid impermeable layer; and
applying a suction force at a fluid conduit of the urine capture device to draw the discharged urine out of the cavity, the fluid conduit providing fluid communication between the urine capture device and a suction source.

19. The method of claim 18, wherein the fluid impermeable layer includes a rear region having the one or more electrical traces secured thereto, a front region positioned generally opposite to the rear region and at least partially defining the opening, and one or more side regions extending between the front region and the rear region, and wherein at least a portion of the fluid permeable member being positioned between the front region and the rear region of the fluid impermeable layer.

20. The method of claim 18, wherein the one or more electrical traces are adhered, molded, or affixed to the inner surface of the fluid impermeable layer, the one or more sensors being connected to an ancillary system configured to receive information from the one or more sensors.
